(19) United States
(12) Patent Application Publication
(10) Pub. No.: US 2008/0228053 A1
(43) Pub. Date: Sep. 18, 2008

(54) METHOD AND SYSTEM FOR CEREBRAL OXYGENATION LEVEL MONITORING

(76) Inventors: Shih-Ping Wang, Los Altos, CA (US); Xuefeng Cheng, Ontario (CA)

Correspondence Address: COOPER & DUNHAM, LLP, 1185 AVENUE OF THE AMERICAS, NEW YORK, NY, 10036, US

(21) Appl. No.: 12/049,784

(22) Filed: Mar. 17, 2008

Related U.S. Application Data
(60) Provisional application No. 60/894,913, filed on Mar. 15, 2007.

(51) Int. Cl. A61B 5/1455

(52) U.S. Cl. 600/340; 600/322

(57) ABSTRACT

Methods, systems, and related computer program products are provided for the reliable measurement of absolute chromophore concentrations in biological tissue, such as those indicative of blood oxygenation levels in the brain, based on infrared optical scanning. A plurality of probe patches is positioned at a respective plurality of locations around the patient's head, each probe patch being positioned against the skin of the head, wherein at least one of the probe patches is positioned where the skin includes active hair follicles. Each probe patch comprises a first infrared source-detector pair having a relatively short source-detector spacing and a second infrared source-detector pair having a relatively long source-detector spacing. Phase-only measurements are acquired for each of a plurality of infrared radiation wavelengths and modulation frequencies for each source-detector pair. Absolute regional chromophore concentrations in the brain are computed based on the phase-only measurements.

### CROSS-REFERENCE TO RELATED APPLICATIONS

This application claims the benefit of Provisional Application No. 60/894,913, filed Mar. 15, 2007, which is incorporated by reference herein. The subject matter of this patent application is related to the subject matter of US 2006/0015021A1, published on Jan. 19, 2006, which is incorporated by reference herein. The subject matter of this patent application is also related to the subject matter of U.S. Ser. No. 12/017,969, filed Jan. 22, 2008, which is incorporated by reference herein.

### FIELD

This patent specification relates generally to the measurement of chromophore concentrations or other properties of biological tissue using information acquired from non-invasive optical scans thereof, such as near infrared optical scans. More particularly, this patent specification relates to cerebral oxygenation level monitoring.

### BACKGROUND AND SUMMARY OF THE DISCLOSURE

The use of near-infrared light as a basis for the measurement of biological properties or conditions in living tissue is particularly appealing because of its relative safety as compared, for example, to the use of ionizing radiation. Various techniques have been proposed for non-invasive near-infrared spectroscopy of biological tissue. Generally speaking, these techniques are directed to detecting the presence and/or measuring the concentrations of one or more chromophores in the biological tissue, such as blood hemoglobin in oxygenated (HbO) and deoxygenated (Hb) states.

One exemplary need for fast, safe imaging of chromophore concentrations in biological tissue, particularly oxygenated hemoglobin levels for the human brain, arises in the context of the millions of surgical procedures performed under general anesthesia every year. One statistic recited in U.S. Pat. No. 5,902,235, which is incorporated by reference herein, is that at least 2,000 patients die each year in the United States alone due to anesthetic accidents, while numerous other such incidents result in at least some amount of brain damage. As discussed therein, certain surgical procedures, particularly of a neurological, cardiac or vascular nature, may require induced low blood flow or pressure conditions, which inevitably involves the potential of insufficient oxygen delivery to the brain. At the same time, the brain is highly intolerant to oxygen deprivation, and brain cells will die within a few minutes if not sufficiently oxygenated. Accordingly, the availability of immediate and accurate information concerning the state of brain oxygen saturation is of critical importance to anesthesiologists and surgeons, as well as other involved medical practitioners. Hospital emergency rooms represent another real-world need for fast, safe measurement of oxygenated hemoglobin levels in the deep human brain.

Pulse oximetry, in which infrared sources and detectors are placed across a thin part of the patient's anatomy such as a fingertip or earlobe, has arisen as a standard of care for all operating room procedures. However, pulse oximetry provides only a general measure of blood oxygenation as represented by the blood passing between the fingertip or earlobe sensor, and does not provide a measure of oxygen levels in vital organs such as the brain. In this sense, the surgeons in the operating room essentially “fly blind” with respect to brain oxygenation levels, which can be a major source of risk for patients (e.g., stroke) as well as a major source of cost and liability issues for hospitals and medical insurers.

Unfortunately, despite their widespread use, doubts have arisen in the medical community about the reliability and measurement validity of known commercially available cerebral oximeters, as discussed in Yoshitani, et. al., “A Comparison of the INVOS 4100 and the NIRO 300 Near-Infrared Spectrophotometers,” Anesthesia and Analgesia, vol. 94, pp. 586-590 (2002). The authors in that study concluded that there was “unacceptable disagreement” in the readings of the INVOS 4100 (Somanetics, Troy, Mich.) and the NIRO 300 (Hamamatsu Photonics, Hamamatsu, Japan) when applied to the same subjects. Although it can be argued that different readings by different devices is not critically harmful, because it is the timewise changes in a reading during surgery that is of actual importance, the Yoshitani paper further found that the relative changes in the readings of the two devices were not equivalent for the same changes in oxygenation. Accordingly, as between the two devices, a scenario is possible in which one of the devices might be indicating a dangerous oxygenation decrease while the other device is not, which is not particularly reassuring.

In another study by Schwarz, et. al., “Cerebral Oximetry in Dead Subjects,” J. Neurosurgical Anesthesiology, Vol. 8, No. 3, pp. 189-193 (1996), an INVOS 3100 device gave readings for six of eighteen dead subjects that had values above the lowest values in a group of healthy adults. Similar problems were identified in Kyutta, et. al., “Extracranial Contribution to Cerebral Oximetry in Brain Dead Patients: A Report of Six Cases,” J. Neurosurgical Anesthesiology, Vol. 11, No. 4, pp. 252-254 (1999). Other major problems can include: high false negative rates; a tendency to provide measurements limited to the skull surface with no definitive brain measurement; a single, relative measurement (whereas oxygenation level problems can happen in multiple brain locations); expense; and limited applicability to other organs. It would be desirable to provide for cerebral oxygenation monitoring having increased accuracy, reliability, and clinical relevance. Other issues arise as would be apparent to one skilled in the art upon reading the present disclosure.

Methods, systems, and related computer program products are provided for the reliable measurement of absolute chromophore concentrations in biological tissue, such as those indicative of blood oxygenation levels in the brain, based on infrared optical scanning. For one preferred embodiment, a plurality of probe patches is positioned at a respective plurality of locations around the patient's head, each probe patch being positioned against the skin of the head, wherein at least one of the probe patches is positioned where the skin includes active hair follicles. Each probe patch comprises a first infrared source-detector pair having a relatively short source-detector spacing and a second infrared source-detector pair having a relatively long source-detector spacing. For each source-detector pair for each of the probes, phase measurements are acquired for each of a plurality of infrared radiation wavelengths and, for each of the plurality of infrared radiation wavelengths, a plurality of modulation frequencies. Based on the phase measurements, a plurality of absolute regional chromophore concentrations in the brain is computed corresponding respectively to each of the probe patches. For one preferred embodiment, the computing of the absolute regional chromophore concentration for each probe patch comprises processing a first subset of the phase measurements associated with the first infrared source-detector pair to compute intermediate results indicative of a radiation propagation property of an area of the patient's skull adjacent to the probe patch. The intermediate results are then processed in conjunction with a second subset of the phase measurements associated with the second infrared source-detector pair to compute the absolute regional chromophore concentration. Preferably, the phase measurements are phase-only measurements, with no intensity attenuation measurements being used in the computing of the absolute regional chromophore concentrations.

### BRIEF DESCRIPTION OF THE DRAWINGS

FIG. 1 illustrates a front view, a top view, and a side view of an arrangement of multiple infrared probe patches disposed around the head of a patient according to a preferred embodiment;

FIGS. 2A and 2B illustrate probe patches for use in the arrangement of FIG. 1;

FIG. 3 illustrates an example of sensor detection range regions for the arrangement of FIG. 1; and

FIG. 4 illustrates an example of sensor detection range regions for the arrangement of FIG. 1.

### DETAILED DESCRIPTION

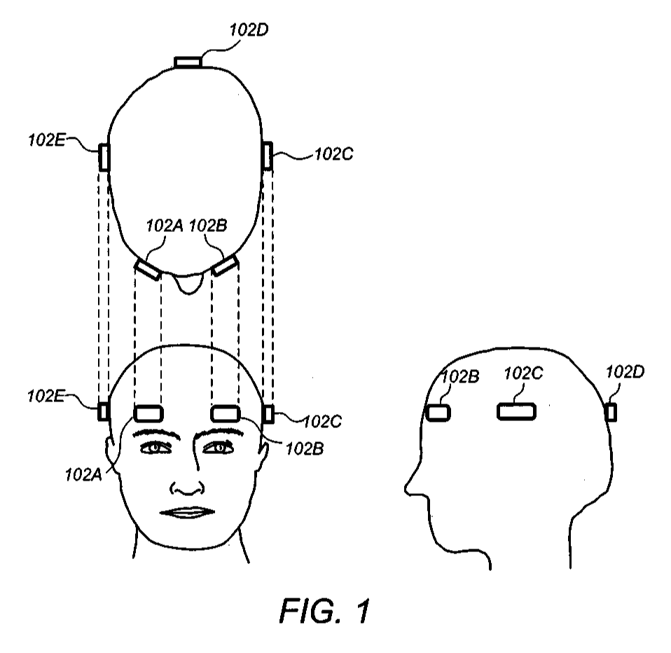

FIG. 1 illustrates an arrangement of multiple probe patches, such as five probe patches 102A-102E, placed around the head, wherein each probe patch is pressed against the skin and/or adhered to the skin using an adhesive. Any of a variety of different physical mechanisms for holding the probe patches 102A-102E in place can be used, including as those described in U.S. Ser. No. 12/017,969, supra. The probe patches may be structurally similar to those discussed in U.S. Pat. No. 5,902,235, supra. However, unlike the teachings of U.S. Pat. No. 5,902,235, supra, one or more of the probe patches can be placed over areas on the head where the skin includes active hair follicles. Preferably, phase-only measurements are used to compute absolute regional chromophore concentrations according to the methods of US 2006/0015021A1, supra. Those phase measurements and related computations have been found to be generally robust against hair follicles or similar obstructions, whereas intensity attenuation measurements can be highly confounded by hair follicles. Notably, although the preferred embodiments are robust against the presence of hair follicles in the skin itself, it is still preferable that any hair growing out of the those follicles be shaved off at locations where the probe patches will be placed.

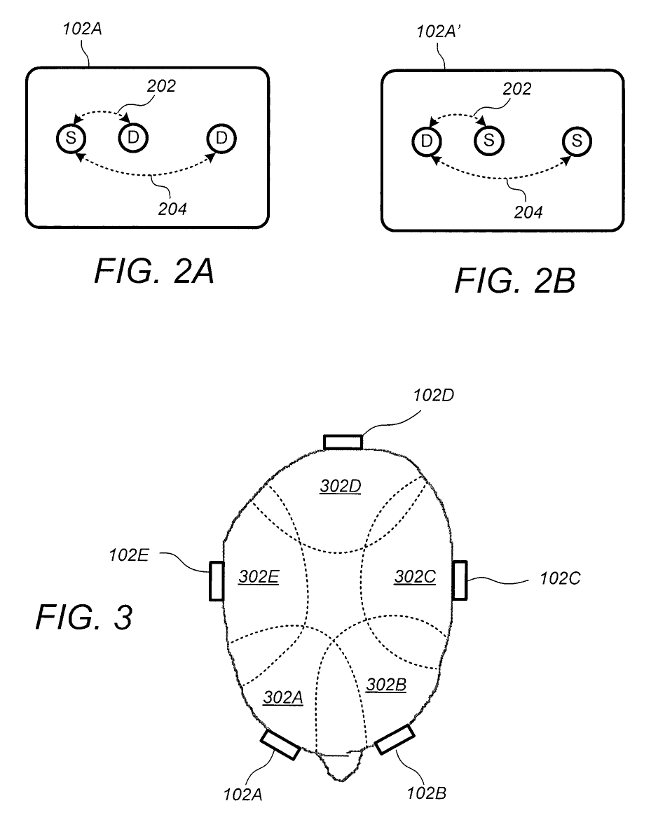

FIGS. 2A and 2B illustrate exemplary probe patches that can be used, each having a first infrared source-detector pair 202 detector having a relatively short source-detector spacing and a second infrared source-detector pair 204 having a relatively long source-detector spacing. The sources “S” and detectors “D” can be similar to those described in US 2006/0015021A1, supra. The detectors can optionally comprise photomultipliers with fiber couplings for very high sensitivity. Preferably, the patches are configured to be disposable for one-time use only, for both sanitary purposes and reliability of results.

The preferred method further comprises acquiring, for each source-detector pair for each of the probes, a phase measurement for each of a plurality of infrared radiation wavelengths and, for each of said plurality of infrared radiation wavelengths, a plurality of modulation frequencies, in a manner similar to that described in US 2006/0015021A1, supra. Based on these phase measurements, a plurality of absolute regional chromophore concentrations in the brain corresponding respectively to each of the probe patches is computed.

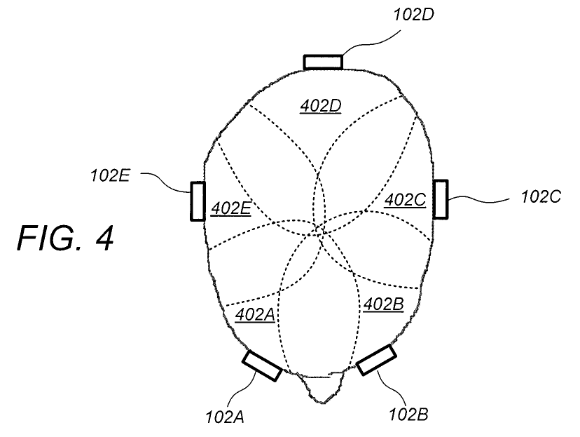

FIG. 3 illustrates examples of sensor detection range regions 302A-302E as associated with the respective probe patches 102A-102E. The regions 302A-302E will generally have some overlap. FIG. 4 illustrates an example according to a preferred embodiment in which sensor detection range regions 402A-402E as associated with the respective probe patches 102A-102E overlappingly extend into deep brain regions.

Preferably, the layer-by-layer computation approach described in US 2006/0015021A1, supra, is used, wherein the attenuation and scattering coefficients are first computed for the layer immediately next to the probe patch, which in this case is for the skull layer and presumed to be uniform, using the phase measurements from the close-together source detector pairs. Then, this knowledge of the attenuation and scattering coefficients for the first layer is used in conjunction with the phase measurements from the farther-apart source-detector pairs to compute the attenuation and scattering coefficients for the next deeper layer, which in this case is the regional areas 302A-302E. See US 2006/0015021A1, supra, at [0041]-[0042]. Stated another way, the regional chromophore concentration for each of the probe patches preferably comprises processing a first subset of the phase measurements, which are associated with the first infrared source-detector pairs 202 (i.e., the close-together pairs) to compute intermediate results indicative of a radiation propagation property of an area of the patient's skull adjacent to the probe patch. Then, these intermediate results are processed in conjunction with the phase measurements from the second infrared source-detector pairs 204 (i.e., the farther-apart ones) to compute the regional chromophore concentrations in the brain across the skull from the probe patch.

For one preferred embodiment, each of the infrared radiation wavelengths can lie between about 600 nm and 900 nm, and each of the carrier modulation frequencies can lie between about 50 MHz and 1 GHz. For one preferred embodiment, each regional chromophore concentration is computed using phase measurements solely from the source-detector pairs of the corresponding probe patch (e.g., the regional chromophore concentration for region 302C only uses measurements from probe patch 102C). For other preferred embodiments, additional phase measurements from additional source-detector pairs are used in computing the regional chromophore concentrations, the additional source-detector pairs being formed from sources and detectors on different ones of the probe patches.

Although the preferred embodiments are generally applicable to a variety of different properties that can be determined for volumetric basis regions for the source-detector pairs, it has been found especially advantageous to use the methods of US 2006/0015021A1, supra, and the resultant measured quantities as subjects for the presently described methods. Thus, for one preferred embodiment, for each source-detector pair, a plurality of phase readings is acquired for different frequencies (e.g., 100 MHz, 120 MHz, and 140 MHz), and then scattering coefficients and attenuation coefficients are computed as described at [0039]-[0040] of US 2006/0015021A1, supra. The oxygenated hemoglobin concentration can then be determined using the scattering coefficients and attenuation coefficients as described in US 2006/0015021A1, supra. Thus provided according to one or more of the preferred embodiments is a 5-channel system capable of definitive, absolute measurement of brain health in the form of cellular oxygen perfusion measurement during operating room procedures, intensive care unit recovery, and in other clinical situations. Advantageously, the non-invasive absolute blood oxygenation monitoring provided according to one or more of the preferred embodiments is readily extendable beyond the brain scenario for measuring oxygen perfusion in the cells of any organ.

For one preferred embodiment, the phase measurements are phase-only measurements, and no intensity attenuation measurements are used in the computing of the regional chromophore concentrations. However, this should not be interpreted as excluding intensity attenuation measurements altogether, and in other embodiments intensity attenuation measurements can also be used, for example, for probe patches on the forehead or on bald areas where there are no hair follicles, or where there is otherwise no adverse affect by virtue of the hair follicles.

By way of example, in another preferred embodiment, a first type of probe patch optimized for combined intensity and phase measurements is used on the forehead or on bald areas where there are no hair follicles, while a second type of probe patch optimized for phase-only measurements is used in other areas where there are hair follicles. As between any two patches of the second type, and as between any pair consisting of a patch of the first type and a patch of the second type, strictly phase-only measurements are taken. However, as between any two patches of the first type, combined intensity and phase measurements are taken.

As used hereinabove, chromophore refers to a substance in a physiological medium which exhibits at least minimum optical interaction with electromagnetic waves transmitting therethrough. Such chromophores may include solvents of a medium, solutes dissolved in the medium, and/or other substances included in such medium. Examples of such chromophores may include, but not limited to, oxygenated hemoglobin, deoxygenated hemoglobin, cytochromes, cytosomes, cytosols, enzymes, hormones, neurotransmitters, chemical or chemotransmitters, proteins, cholesterols, apoproteins, lipids, carbohydrates, blood cells, water, and other optical materials present in the animal or human cells, tissues or body fluid. Chromophores may also include extra-cellular substances which may be injected into the medium for therapeutic and/or imaging purposes or for creating interaction with electromagnetic waves. Typical examples of such chromophores may include, but not limited to, dyes, contrast agents, and other image-enhancing agents, each of which may be designed to exhibit optical interaction with electromagnetic waves having wavelengths in a specific range to be disclosed below. Hemoglobin refer to oxygenated hemoglobin (i.e., HbO) and/or deoxygenated hemoglobin (i.e., Hb) or sum thereof. As used herein, “property” of a chromophore (or hemoglobin) may be an intensive property such as their concentrations, a sum of such concentrations, a difference therebetween, and a ratio thereof. Such property may also be extensive property such as, e.g., volume, mass, weight, volumetric flow rate, and mass flow rate of the chromophores (or hemoglobins).

Whereas many alterations and modifications of the embodiments will no doubt become apparent to a person of ordinary skill in the art after having read the foregoing description, it is to be understood that the particular embodiments shown and described by way of illustration are in no way intended to be considered limiting. By way of example, although detection and volumetric imaging of blood oxygenation levels is described in several of the preferred embodiments above, the described methods have also been found useful for measurement of carbon dioxide (CO₂) levels in the brain and elsewhere. In a similar way as O₂ delivery in biological system, CO₂ partially resolves or dissolves in the blood, binds with hemoglobin to form Hb_CO₂, and eventually is released in the lung during gas exchange. Hb_CO2 has a distinguished absorption coefficient and its concentration can be obtained through similar spectroscopic measurement and principles as the oxygenated hemoglobin determination described in US 2006/0015021A1, supra. Through measuring Hb_CO₂, the PH+ value can be derived as well as the CO₂ partial pressure inside cells.

By way of further example, in other preferred embodiments one or more methods of U.S. Pat. No. 6,397,099, which is incorporated by reference herein, can be used to position/secure the probe patches to the patient's head. By way of further example, although the use of five (5) probe patches has been found particularly advantageous, other numbers of probe patches (for example, between 4 and 8 probe patches, or other numbers in other preferred embodiments) can be used without necessarily departing from the scope of the present teachings. By way of still further example, in other preferred embodiments there are many source-detector pairs on each probe patch for enabling the computation of detailed two- or three-dimensional images of the brain tissue chromophore levels in the regional areas across the skull from each respective probe patch. By way of even further example, although absolute chromophore concentrations (for example, absolute concentrations of oxygenated hemoglobin HbO and deoxygenated hemoglobin Hb) are preferable because they can eliminate problems analogous to the dead-subject problems discussed above the background section, it is not necessarily outside the scope of the present teachings to compute relative oxygenation levels only, such as the relative percentage of oxygenated hemoglobin to total hemoglobin. Thus, reference to the details of the preferred embodiments are not intended to limit their scope.

What is claimed is:

1. A method for measuring a plurality of absolute regional chromophore concentrations in a brain of a patient, comprising:
placing a plurality of probe patches at a respective plurality of locations around the patient's head, each probe patch being positioned against the skin of the head, at least one of said probe patches being positioned where the skin includes active hair follicles, each probe patch having a first infrared source-detector pair having a relatively short source-detector spacing and a second infrared source-detector pair having a relatively long source-detector spacing;
acquiring, for each source-detector pair for each of said probes, a phase measurement for each of a plurality of infrared radiation wavelengths and, for each of said plurality of infrared radiation wavelengths, a plurality of modulation frequencies; and
computing based on said phase measurements a plurality of absolute regional chromophore concentrations in the brain corresponding respectively to said plurality of probe patches.

2. The method of claim 1, wherein said computing the absolute regional chromophore concentration for each of said probe patches comprises:
processing a first subset of said phase measurements associated with said first infrared source-detector pair to compute intermediate results indicative of a radiation propagation property of an area of the patient's skull adjacent to said probe patch; and
processing said intermediate results in conjunction with a second subset of said phase measurements associated with said second infrared source-detector pair to compute said absolute regional chromophore concentration.

3. The method of claim 2, wherein said phase measurements are phase-only measurements, and wherein no intensity attenuation measurements are used in said computing of said absolute regional chromophore concentrations.

4. The method of claim 3, wherein each of said infrared radiation wavelengths lies between about 600 nm and 900 nm, and wherein each of said carrier modulation frequencies is between about 50 MHz and 1 GHz.

5. The method of claim 3, wherein said plurality of probe patches consists of a first probe patch positioned above one eye of the patient, a second probe patch positioned above the other eye of the patient, a third probe patch positioned above one ear of the patient, a fourth probe patch positioned above the other ear of the patient, and a fifth probe patch positioned at the back of the patient's head.

6. The method of claim 3, wherein said plurality of probe patches comprises between 4 and 8 probe patches so placed around the patient's head.

7. The method of claim 3, further comprising measuring an additional absolute regional chromophore concentration in the brain, the additional absolute regional chromophore concentration corresponding to a pairing between a first one and a second one of said plurality of probe patches, comprising:
acquiring, for a third source-detector pair formed by a source on one of said first and second probe patches and a detector on the other of said first and second probe patches, phase-only measurements for each of the plurality of infrared radiation wavelengths and modulation frequencies; and
processing said phase-only measurements for said third source-detector pair in conjunction with the phase-only measurements for the first and second source-detector pairs of each of said first and second probe patches to compute said additional absolute regional chromophore concentration.

8. A system for facilitating patient monitoring, comprising:
a plurality of probe patches configured and adapted for placement at a respective plurality of locations around the patient's head such that each probe patch is positioned against the skin of the head and such that at least one of said probe patches is positioned where the skin includes active hair follicles, wherein each probe patch comprises a first infrared source-detector pair having a relatively short source-detector spacing and a second infrared source-detector pair having a relatively long source-detector spacing;
a processor configured to receive, for each source-detector pair for each of said probes, a phase measurement for each of a plurality of infrared radiation wavelengths and, for each of said plurality of infrared radiation wavelengths, a plurality of modulation frequencies, said processor being further configured to compute based on said phase measurements a plurality of absolute regional chromophore concentrations in the brain corresponding respectively to said plurality of probe patches; and
a display output coupled to said processor for displaying said absolute regional chromophore concentrations to a viewer.

9. The system of claim 8, wherein said processor computes the absolute regional chromophore concentration for each of said probe patches by (i) processing a first subset of said phase measurements associated with said first infrared source-detector pair to compute intermediate results indicative of a radiation propagation property of an area of the patient's skull adjacent to said probe patch, and (ii) processing said intermediate results in conjunction with a second subset of said phase measurements associated with said second infrared source-detector pair to compute said absolute regional chromophore concentration.

10. The system of claim 8, wherein said phase measurements are phase-only measurements, and wherein no intensity attenuation measurements are used by said processor in said computing of said absolute regional chromophore concentrations.

11. The system of claim 10, wherein each of said infrared radiation wavelengths lies between about 600 nm and 900 nm, and wherein each of said carrier modulation frequencies is between about 50 MHz and 1 GHz.

12. The system of claim 10, wherein said plurality of probe patches consists of a first probe patch positioned above one eye of the patient, a second probe patch positioned above the other eye of the patient, a third probe patch positioned above one ear of the patient, a fourth probe patch positioned above the other ear of the patient, and a fifth probe patch positioned at the back of the patient's head.

13. The system of claim 10, wherein said plurality of probe patches comprises between 4 and 8 probe patches for placement around the patient's head.

14. The system of claim 10, the system being further configured to measure an additional absolute regional chromophore concentration in the brain, the additional absolute regional chromophore concentration corresponding to a pairing between a first one and a second one of said plurality of probe patches, wherein said processor is further configured (i) to receive, for a third source-detector pair formed by a source on one of said first and second probe patches and a detector on the other of said first and second probe patches, phase-only measurements for each of the plurality of infrared radiation wavelengths and modulation frequencies, and (ii) to process said phase-only measurements for said third source-detector pair in conjunction with the phase-only measurements for the first and second source-detector pairs of each of said first and second probe patches to compute said additional absolute regional chromophore concentration.

15. A computer program product tangibly stored on a computer-readable medium for facilitating non-invasive patient monitoring, comprising:
computer code for receiving phase measurements corresponding to each of a plurality of infrared source-detector pairs disposed on each of a plurality of probe patches, said plurality of probe patches being positioned against the skin of a patient's head at respective locations therearound including at least one location where the skin includes active hair follicles, said plurality of infrared source-detector pairs on each said probe patch including a first source-detector pair having a relatively short source-detector spacing and a second source-detector pair having a relatively long source-detector spacing, said phase measurements for each source-detector pair corresponding to a plurality of infrared radiation wavelengths and, for each infrared radiation wavelength, a plurality of modulation frequencies;
computer code for computing based on said phase measurements a plurality of absolute regional chromophore concentrations in the brain corresponding respectively to said plurality of probe patches; and
computer code for outputting said plurality of absolute regional chromophore concentrations to a viewable display.

16. The computer program product of claim 15, wherein said computer code for computing the absolute regional chromophore concentration for each of said probe patches comprises:
computer code for processing a first subset of said phase measurements associated with said first infrared source-detector pair to compute intermediate results indicative of a radiation propagation property of an area of the patient's skull adjacent to said probe patch; and
computer code for processing said intermediate results in conjunction with a second subset of said phase measurements associated with said second infrared source-detector pair to compute said absolute regional chromophore concentration.

17. The computer program product of claim 16, wherein said phase measurements are phase-only measurements, and wherein no intensity attenuation measurements are used in said computing of said absolute regional chromophore concentrations.

18. The computer program product of claim 17, wherein each of said infrared radiation wavelengths lies between about 600 nm and 900 nm, and wherein each of said carrier modulation frequencies is between about 50 MHz and 1 GHz.

19. The computer program product of claim 17, wherein said plurality of probe patches consists of a first probe patch positioned above one eye of the patient, a second probe patch positioned above the other eye of the patient, a third probe patch positioned above one ear of the patient, a fourth probe patch positioned above the other ear of the patient, and a fifth probe patch positioned at the back of the patient's head.

20. The computer program product of claim 17, further-comprising computer code for computing an additional absolute regional chromophore concentration in the brain, the additional absolute regional chromophore concentration corresponding to a pairing between a first one and a second one of said plurality of probe patches, comprising:
computer code for receiving, for a third source-detector pair formed by a source on one of said first and second probe patches and a detector on the other of said first and second probe patches, phase-only measurements for each of the plurality of infrared radiation wavelengths and modulation frequencies; and
computer code for processing said phase-only measurements for said third source-detector pair in conjunction with the phase-only measurements for the first and second source-detector pairs of each of said first and second probe patches to compute said additional absolute regional chromophore concentration.
